(19) United States
(12) Patent Application Publication
(10) Pub. No.: US 2005/0197529 A1
(43) Pub. Date: Sep. 8, 2005

(54) Endoscopic mouthpiece

(75) Inventors: Yoshio Hoshihara, Tokyo (JP); Tetsuo Udagawa, Tokyo (JP); Katsuya Kikuchi, Saitama (JP); Mamoru Machiya, Saitama (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: FUJINON CORPORATION, Saitama-shi (JP)

(21) Appl. No.: 11/072,540

(22) Filed: Mar. 7, 2005

(30) Foreign Application Priority Data
Mar. 8, 2004 (JP) 2004-063618

(51) Int. Cl. A61B 1/24

(52) U.S. Cl. 600/114

(57) ABSTRACT

A mouthpiece for use in upper endoscopy, including a main body of generally tubular shape internally providing a passage for receiving an endoscopic insertion tube and having a teeth support portion on the outer periphery of the passage portion. The internal passage in the main body is provided with a partition plate, thereby dividing the internal passage into an upper passage for introduction of an endoscopic insertion tube and a lower passage adapted to receive and hold a patient's tongue in such a way to restrict its movements.

### BACKGROUND OF THE INVENTION

1. Field of the Art

This invention relates to an endoscopic mouthpiece, and more particularly to a mouthpiece for use in upper endoscopy, for example, for use in endoscopic examination of an upper digestive tract to prevent a patient from biting on an insertion tube of an endoscope with his or her teeth when the insertion tube is orally introduced into an intracavitary site of interest via the throat.

2. Prior Art

Endoscopes for upper digestive tracts, such as gastroscopes and duodenoscopes, are equipped with an elongated insertion tube of flexible construction, which is freely bendable along a path of insertion and orally inserted through the throat and esophagus as far as an intracavitary site to be examined. At the time of insertion, it is necessary to hold the patient's oral cavity forcibly in an open state since otherwise the patient would bite on the insertion tube, causing damages to outer surfaces of the insertion tube and in some cases making the insertion of the endoscope difficult. Therefore, it has been the general practice to set a mouthpiece in the patient's mouth thereby to keep the patient's teeth out of direct contact with an insertion tube of an endoscope.

Generally, an endoscopic mouthpiece to be placed between patient's teeth is formed in a tubular shape and internally provided with a tunnel-like passage to receive an endoscopic insertion tube. In order to let a patient hold the mouthpiece stably in his or her mouth, the mouth piece is not formed in a simple tubular shape, but it is usually flared toward front and rear ends from a minimum diameter portion to be held between patient's teeth. It follows that, even if an external force is applied on the mouthpiece in an outward or inward direction, it would not easily get unstable or fall off the patient's mouth.

In this connection, when an endoscopic insertion tube is introduced into the throat of a patient via a mouthpiece which is placed in the patient's mouth, the patient feels vomiting sensations at the time the insertion tube is passed through the throat. The vomiting sensations are aroused as the insertion tube is pushed up and brought into contact with the upper jaw by movements of the patient's tongue. Therefore, it is necessary for the mouthpiece to be able to hold the patient's tongue down while an endoscopic insertion tube is passed through the throat. For this purpose, attempts have been made to provide a plate-like tongue holder on an endoscopic mouthpiece body, for example, as disclosed in Japanese Laid-Open Patent Application S55-101.

Even if a tongue holder plate is extended from a mouthpiece body as proposed by the prior art just mentioned, its length is limited. If the tongue holder on the mouthpiece is extended to such a degree as to hold a root portion of a tongue, the tongue holder can cause constriction of the throat by pushing down the tongue at the time of placing the mouthpiece in the mouth of a patient. As a consequence, the long tongue holder can make it difficult to introduce an endoscopic insertion tube through patient's throat, and can cause greater pains on the part of the patient, increasing the vomiting sensations all the more. Therefore, a tongue holder of an inappropriate length can have the opposite effects, that is, have no effects in decreasing the vomiting sensations a patient feels when taking an endoscopic mouthpiece in the mouth.

### SUMMARY OF THE INVENTION

In view of the foregoing situations, it is an object of the present invention to provide an endoscopic mouthpiece to be placed in a patient's mouth in upper endoscopy, the mouthpiece being so arranged as to permit smooth passage of an endoscopic insertion tube without causing vomiting sensations to the patient.

It is another object of the present invention to provide an endoscopic mouthpiece which is so arranged as to let an endoscopic insertion tube pass through the throat of a patient smoothly and easily without causing pains on the part of the patient.

In accordance with the present invention, in order to achieve the above-stated objectives, there is provided an endoscopic mouthpiece having a main body generally of a tubular shape internally providing a passage for an endoscopic insertion tube and externally providing a teeth support portion on outer periphery of the passage, characterized in that said mouthpiece comprises: a partition plate provided in and across the passage to divide same into a first or upper passage and a second or lower passage; the upper passage being adapted to serve as a passage of an endoscopic insertion tube; and the lower passage being adapted to receive a patient's tongue in such a way as to restrict movements of the tongue.

The mouthpiece is arranged to restrict movements of patient's tongue in an effective manner as soon as it is stuck into the second passage under the first passage, particularly minimizing and suppressing upward movements of the tongue which tends to push up an endoscopic insertion tube. In a preferred form of the present invention, for stabilization of the tongue which is trapped in the second lower passage, a tongue holder is extended from the partition plate beyond a proximal stopper end of the mouthpiece. According to the general procedure of upper endoscopy, a patient lies on her or his left side during an examination. Therefore, it is desirable to form the tongue holder portion in a smaller width as compared with the second lower passage and to make arrangements such that a tongue holder portion of a reduced width is inclined downward when a patient takes a lying posture. In this case, part of the patient's tongue which cannot be received in the second passage can be accommodated in open spaces on the opposite side of the tongue holder portion. For this purpose, the width of the tongue holder portion is gradually and continuously reduced toward a proximal free end by cutting one side of the tongue holder portion obliquely toward the other side. The patient tongue in the second passage is displaced toward an open space on the lower side, and as a result a space is opened on the upper side of the second passage to ease breathing of the patient. Further, according to another preferred form of the present invention, a guide rib is provided on and along the partition plate and the tongue holder portion on the side of the first upper passage thereby to guide an endoscopic insertion tube through a center portion of the first passage in an assured manner. Further, if necessary, a liquid injection hole can be bored through the partition plate for injection of an anesthetic agent or other medicinal liquids.

The above and other objects, features and advantages of the present invention will become apparent from the following particular description of the invention, taken in conjunction with the accompanying drawings which show by way of example some preferred embodiments of the invention. Needless to say, the present invention should not be construed as being limited to particular forms which are shown in the drawings.

### BRIEF DESCRIPTION OF THE DRAWINGS

In the accompanying drawings:

FIG. 1 is a schematic sectional view of an endoscopic mouthpiece which is placed in the mouth of a patient and an endoscopic insertion tube being introduced into the throat of the patient through the mouthpiece;

FIG. 2 is a schematic perspective view of the mouthpiece shown in FIG. 1;

FIG. 3 is a schematic plan view of the mouthpiece shown in FIG. 1;

FIG. 4 is a schematic back view of the mouthpiece shown in FIG. 1

FIG. 5 is a right-hand side view of the mouthpiece shown in FIG. 1;

FIG. 6 is a schematic illustration explanatory of the manner how a patient's tongue is held in a restricted state by the mouthpiece;

FIG. 7 is a view similar to FIG. 2 but showing a modification of the mouthpiece;

FIG. 8 is a view similar to FIG. 3 but showing another modification of the mouthpiece; and

FIG. 9 is a view similar to FIG. 4 but showing still another modification of the mouthpiece.

### DESCRIPTION OF PREFERRED EMBODIMENTS

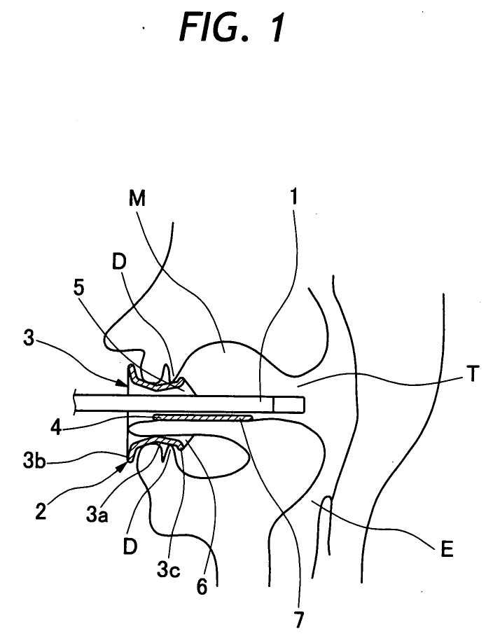

Hereafter, with reference to the accompanying drawings, the present invention is described more particularly by way of its preferred embodiments. Referring first to FIG. 1, there are shown an endoscopic mouthpiece according to the present invention, and an endoscopic insertion tube which is being introduced into the throat of a patient through the mouthpiece.

In FIG. 1, indicated at 1 is an insertion tube of an endoscope, and at 2 a mouthpiece. The mouthpiece 2 is placed in the mouth of a patient in such a way that it is gripped between the teeth of the patient. Through the mouthpiece 2, the endoscopic insertion tube 1 is introduced into the esophagus E via the throat T.

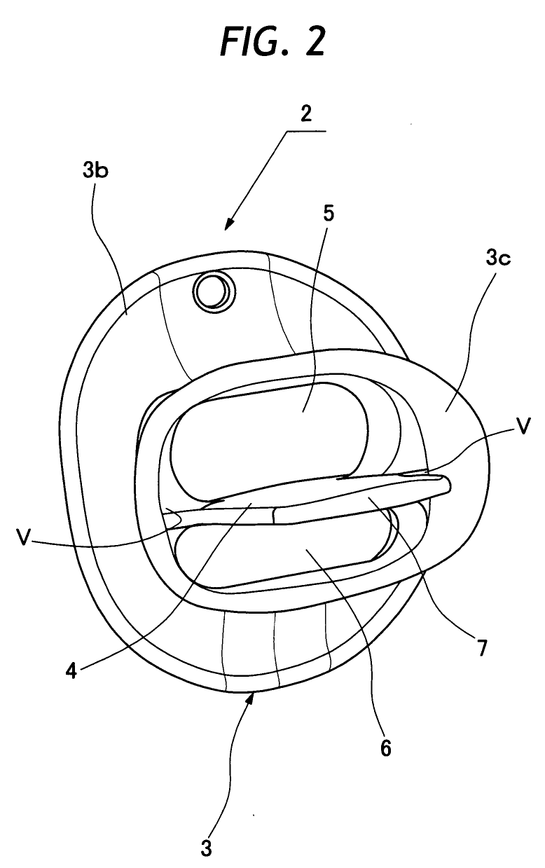

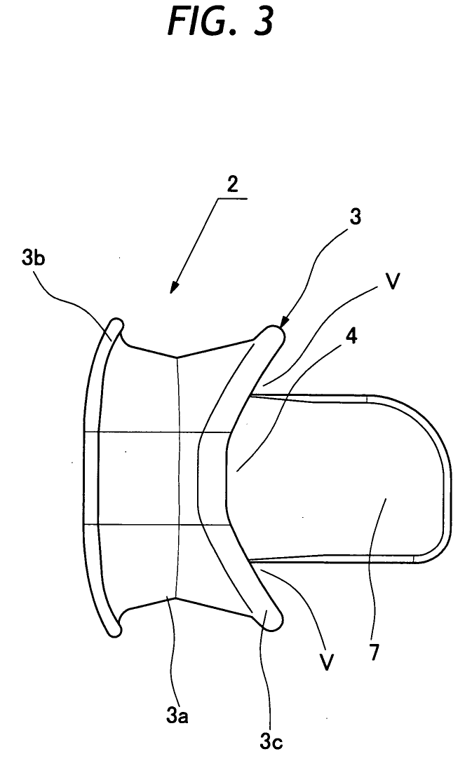

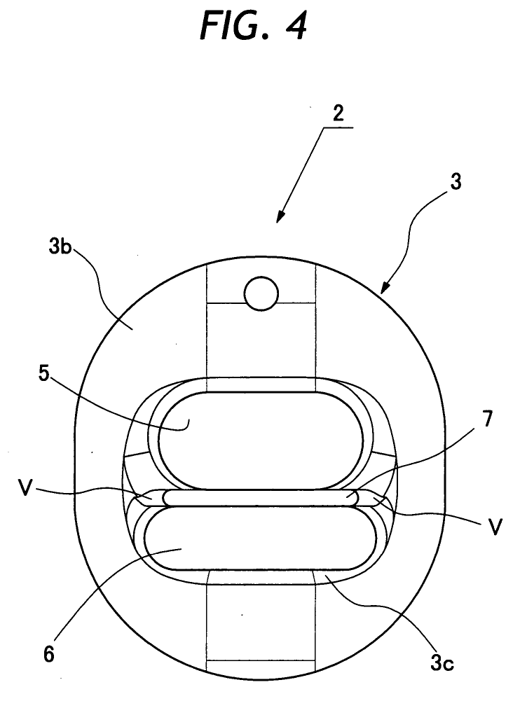

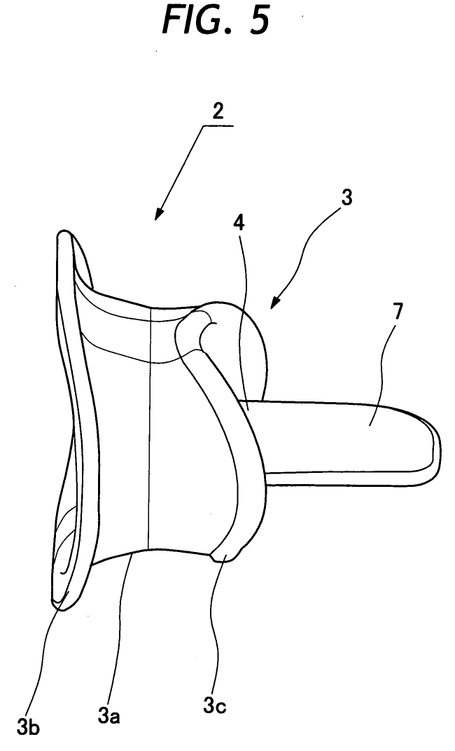

The mouthpiece 2 has a main body 3 which is formed generally in a tubular shape. As shown in FIGS. 2 to 5, the mouthpiece 2 is provided with a teeth support portion 3a at a longitudinally intermediate portion of the main body 3, and, a fore end portion of the main body 3 forward of the teeth support portion 3a, in other words, a fore end portion which is exposed on the front side of patient's lips is flared in a funnel-like shape to provide an entrance 3c to the mouthpiece 2. The other rear end which is placed in the mouth M is moderately flared to provide a stopper portion 3c. Thus, the main body 3 of the mouthpiece 2 is formed in a narrowest diameter at the teeth support portion 3a. Therefore, when the mouthpiece 2 is placed in a patient's mouth M, the teeth support portion 3a is firmly gripped between patient's teeth D to hold the mouthpiece 2 stably in the mouth M in an immovable state particularly in inward and outward directions.

More specifically, between the flared front and rear ends 3a and 3c, the tubular main body 3 of the mouthpiece 2 is formed in an elliptic shape in cross section, and a partition plate 4 is provided across an elliptic internal passage approximately at an intermediate position in the direction of the long axis of the elliptic passage, dividing the elliptic passage into upper and lower passages 5 and 6. Namely, the elliptic internal passage within the main body 3 of the mouthpiece 2 is divided by the partition plate 4, into a first passage 5 which is located on the upper side and a second passage 6 which is located on the lower side. The first passage 5 is provided as a passage for an endoscopic insertion tube 1 while the second passage 6 is provided as a pocket for receiving a patient's tongue through the inner stopper end 3c of the mouthpiece 2. The partition plates 4 which defines the first and second passages 5 and 6 in the mouthpiece 2 is projected on the proximal side of the stopper end 3c over a predetermined extension length to provide a tongue holder portion 7 at the innermost free end of the partition plate 4.

In this instance, at an intermediate portion between the funnel-like entrance end 3b and the stopper end 3c, the partition plate 4 is connected to inner surfaces of the tubular main body 3 of the mouthpiece 2 over a predetermined length depending upon the size of the mouthpiece 2, but disconnected from inner surfaces of the main body 3 from a transitional portion leading to the tongue holder portion 7 at the innermost free end. Therefore, a gap space V is left at each side of the partition plate 4 over a predetermined length. The tongue holder portion 7 needs to have a certain length to function as a tongue holder, but should be in a length which falls short of the throat T.

In use, prior to insertion of an endoscope, the mouthpiece 2 of the above-described construction is placed in the mouth of a patient who is going to take an examination by upper endoscopy. The mouthpiece 2 can be set in position by pushing the main body 3 of the mouthpiece 2 into the mouth M until the teeth support portion 3a is gripped between teeth of the patient. At this time, the tongue holder portion 7 which is provided as an extension of the partition plate 4 is allowed to advance to a deeper portion of the mouth M. By sticking out the tongue under the tongue holder portion 7, the patient's tongue is placed and trapped in the second passage 6 on the lower side of the first passage 5 of the mouthpiece 2. In so doing, the patient urged to stick the tip end of his or her tongue into the second passage 6 as much as possible. The sectional area of the tongue increases toward its root portion, getting thicker beyond the sectional area of the second passage 6. However, since the gap spaces V are provided on the opposite sides of the partition plate 4 an inner or rear portion of which is disconnected and set apart from inner surfaces of the diverging stopper end 3c, opposite side portions of the tongue are received in the gap spaces V. As a consequence, despite increases in sectional area, the tongue can be received in the second passage 6 in an unstrained state and over a sufficient length for restricting movements of the tongue. Behind the second passage 6, the tongue lies under the tongue holder portion 7 on the main body 3 of the mouthpiece. Therefore, the tongue is effectively restricted of movements, particularly restricted of movements in upward, downward, rightward and leftward directions.

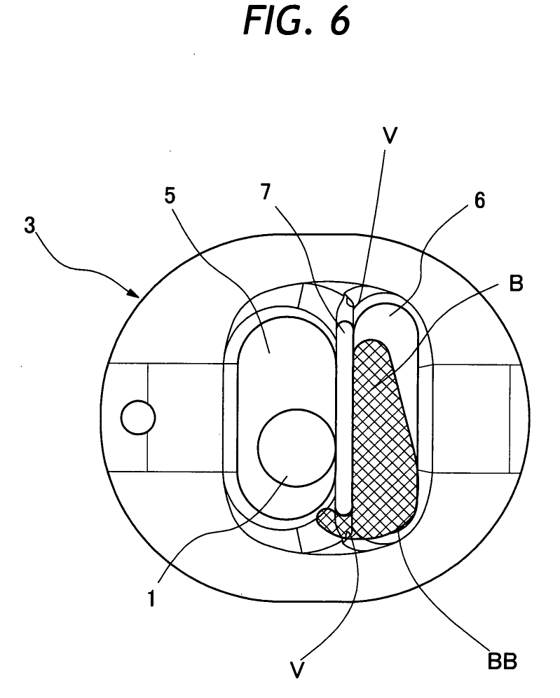

At the time of an endoscopic examination, it is an ordinary procedure for a patient to lie on one's side. Therefore, the gravity constantly acts on the tongue of the patient. If movements of the tongue is completely restricted by the second passage 6, it could cause a great burden to the patient, increasing the tension on the part of the patient to a considerable degree. Therefore, if an endoscopic examination takes a long time, the patient would be fatigued considerably. However, since the void spaces V are opened in the second passage 6, the patient's tongue B can move into the void spaces V as shown in FIG. 6, without possibilities of interference with the endoscopic insertion tube 1 in the first passage 5 of the mouthpiece. Accordingly, on the back side BB, the patient's tongue B is supported by broad wall surfaces of the mouthpiece from the second passage 6 to a position past the void space V. Thus, the tongue B is supported in a stabilized state and with a certain degree of freedom of movements to lessen tension and fatigue on the part of the patient to a significant extent.

After placing the mouthpiece in the patient's mouth, the endoscopic insertion tube 1 is introduced into the first passage 5 of the mouthpiece 2. The insertion tube 1 is kept out of contact with the tongue until it is inserted beyond the tongue holder portion 7. That is, up to this point, the tongue receives no stimulations from the insertion tube, and remains substantially in a motionless state. When the endoscopic insertion tube 1 is pushed in toward the throat T past the tongue holder portion 7, an inward pushing force is exerted on the throat T. However, since the throat T can be passed within a short distance and the tip end of the tongue is in a forcibly restricted state against movements which would otherwise be aroused by stimulative contact with the insertion tube 1, the root portion of the tongue remains motionless instead of being pushed up toward the upper jaw by the endoscopic insertion tube 1. Once the endoscopic insertion tube 1 passes through the throat T, there is little possibility of the tongue being stimulated by inward movements of the endoscopic insertion tube 1, and vomiting sensations and pains on the part of the patient drop to a minimal level. Accordingly, the endoscopic insertion tube 1 can be smoothly introduced into a body cavity of interest free of interferences by the patient's tongue.

According to the procedures of upper endoscopy, the patient is put on a bed to lie on one side, normally to lie on left side during an examination. Therefore, if the endoscopic insertion tube 1 is introduced straight into the first passage 5 on the main body 3 of the mouthpiece 2 which is placed in the mouth of the lying patient, the insertion tube 1 tends to advance toward a lower corner portion of the first passage 5, namely, to advance along a lower curved portion of the first passage 5. In such a case, maneuverability of the insertion tube 1 can be degraded by its sliding movements, causing difficulties in sniping the fore end of the insertion tube 1 at the esophagus E from the throat T.

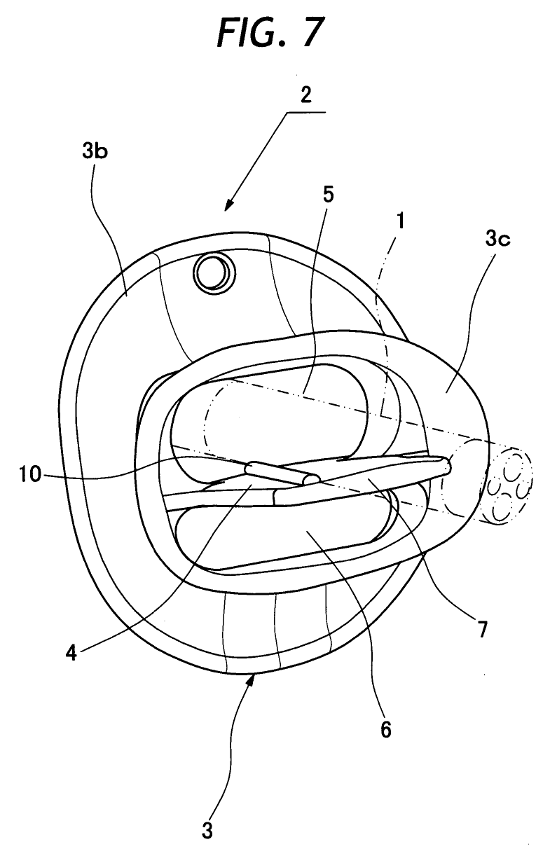

In order to overcome the difficulties just mentioned, in a modification shown in FIG. 7, a guide rib 10 is provided on the partition plate 4 on the side of the first passage 5 of the mouthpiece 2 and extended as far as the tongue holder portion 7. In this instance, arrangements are made such that top end of the guide rib 10 is spaced from inner wall on the upper side of the first passage 5 by a gap space which is narrower than the outside diameter of the insertion tube 1. If the guide rib 10 is arranged in this manner, the endoscopic insertion tube 1 is guided along the guide rib 10 and passed substantially through a center portion of the first passage 5 as indicated in phantom in FIG. 7. Accordingly, thanks to reduction of resistance to sliding movements, it becomes easier to control movements of the endoscopic insertion tube 1 in sniping at the throat T.

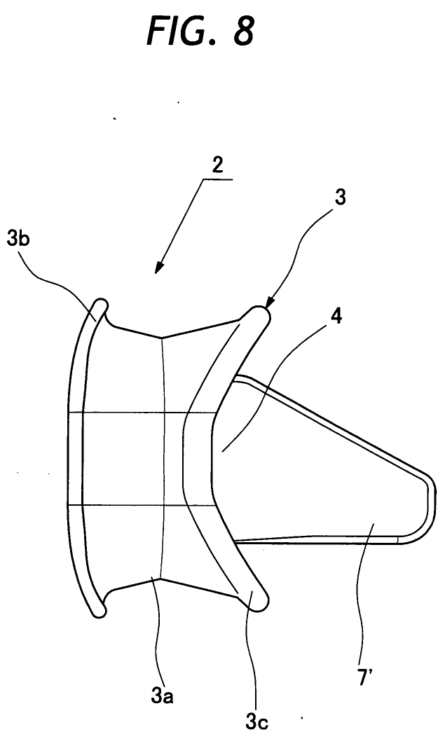

Further, when the mouthpiece 2 is placed in the mouth of a patient, the patient's tongue in the second passage 6 is displaced to the lower side or to the left side of the second passage 6. In such a state, an upper portion of the tongue holder portion 7 is not necessarily required to hold the tongue, and, if desired, an upper portion may be cut off to provide a tongue holder portion 7′ of an approximately triangular shape as shown in FIG. 8. In this case, as the patient's tongue is stuck into the second passage 6, it is displaced to the lower side of the passage 6 to open up more or less a space in an upper portion of the second passage 6, which can serve as a breathing hole to ease the breathing of the patient.

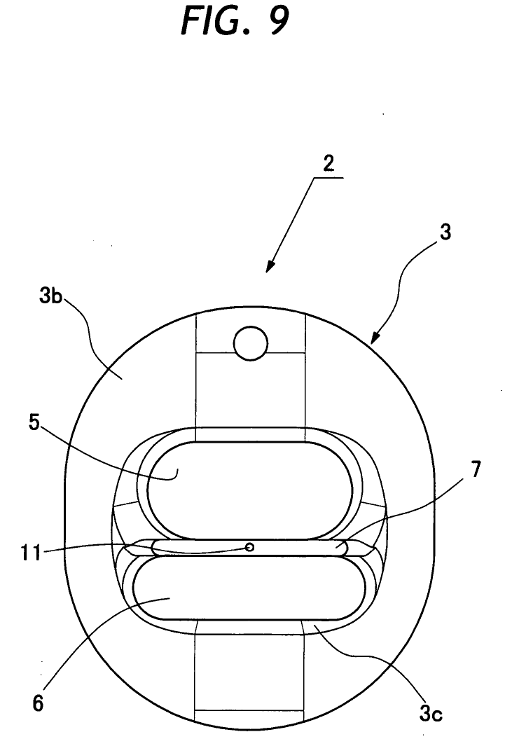

Further, as shown in FIG. 9, a liquid injection hole 11 can be bored through the partition plate 4 and the tongue holder portion 7 of the mouthpiece 2 for use in injecting an anesthetic agent or other medicinal liquid into the mouth or toward the throat T.

1. An endoscopic mouthpiece having a main body generally of a tubular shape internally providing a passage for an endoscopic insertion tube and externally providing a teeth support portion on outer periphery of said passage, characterized in that said mouthpiece comprises:
a partition plate provided in and across said passage to divide same into an upper passage and a lower passage;
said upper passage being adapted to serve as a passage of an endoscopic insertion tube; and
said lower passage being adapted to receive a patient's tongue in such a way as to restrict movements of the tongue.

2. An endoscopic mouthpiece as defined in claim 1, wherein said main body is formed in a smallest diameter at said teeth support portion, and provided with a funnel-like flared end formed contiguously on the front side of said passage as an entrance of an endoscopic insertion tube and a moderately flared end formed contiguously on the proximal side of said teeth support portion as a stopper portion to be placed in a patient's mouth.

3. An endoscopic mouthpiece as defined in claim 1, wherein said main body of said mouthpiece is formed in an elliptic shape in cross section, and said partition plate is located along long axis of an elliptic cross-sectional area in dividing said passage into an upper passage and a lower passage.

4. An endoscopic mouthpiece as defined in claim 1, further comprising a tongue holder portion provided by extending said partition plate rearward beyond said stopper portion.

5. An endoscopic mouthpiece as defined in claim 4, wherein said tongue holder portion is extended rearward from said partition plate in a width narrower than said second passage.

6. An endoscopic mouthpiece as defined in claim 4, wherein one side of said tongue holder portion is inclined toward the other side such that width of said tongue holder portion is gradually reduced toward a rear distal end.

7. An endoscopic mouthpiece as defined in claim 1, wherein a guide rib is provided at least on a surface of said partition plate on the side of said first passage to orient an endoscopic insertion tube in a right direction.

8. An endoscopic mouthpiece as defined in claim 7, wherein a spacing between top end of said guide rib on said partition plate and ceiling wall of said first passage is narrower than outside diameter of said endoscopic insertion tube.

9. An endoscopic mouthpiece as defined in claim 1, further comprising a liquid injection hole bored through said partition plate and tongue holder portion of said mouthpiece.
